(19) United States
(12) Patent Application Publication
(10) Pub. No.: US 2016/0186111 A1
(43) Pub. Date: Jun. 30, 2016

(54) MUNICIPAL WASTE WATER TREATMENT PLANT/PROCESS WITH BY PRODUCT AND DRINKING WATER RECYCLE

(71) Applicant: Thomas Benton Young, III, North Stonington, CT (US)

(72) Inventor: Thomas Benton Young, III, North Stonington, CT (US)

(21) Appl. No.: 14/392,035

(22) PCT Filed: Dec. 29, 2014
(86) PCT No.: PCT/US14/00224

§ 371 Date: Nov. 6, 2015

(51) Int. Cl. C12M 1/107; C02F 1/44; C02F 3/28; C02F 11/04; C12M 1/12; C12M 1/02

(52) U.S. Cl. CPC C12M 21/04; C12M 37/02; C12M 27/00; C02F 3/28; C02F 11/04; C02F 1/444

(57) ABSTRACT

A domestic waste water plant/process design to replace the century old Activated Sludge process that uses modern biochemical engineering equipment allowing removal of bactria of influent waste water by serial separation of solids in stages of clarification at one, higher G's in a continuous splitting bowl centrifuge feeding solids to an anaerobic digester and centrate to a final micro-filtration through an absolute one micron pore size ceramic membrane to a aerobic continuous bioreactor with high energy input/oxygen transfer rates and selected microbes suitable for animal/fish high protein feed of far smaller volume than AS bioreactors having a sidearm loop for microbe retention/liquid purge for short retention times necessary in the bioreactor.

The first bioreactor is an anaerobic digestion of cellulosic solids from the primary clarifier by methane producing anaerobes gives fuel and organic fertilizer. The second bioreactor produces algae or yeast for animal/fish feed by metabolizing organics, nitrogen and phosphorous in the centrifuged, filtered overflow of the clarifier by aerobic fermentation of pure selected cultures in a side arm fermenter where filtrate is purged through a retaining micro-filter and cells are concentrated. The bacteria free filtrate of the purge is further purified in a UF/RO unit of hollow fiber or spiral wound design with molecular weight cutoff similar to desalination units to produce microbe and virus free drinking water to be recycled to the municipal water supply conserving water in areas of limited supply or dense population.

In the last 50 years the earth's population has doubled. This has put a great demand for her yield of food and water. Ground water levels are receding as monitored by NASA globally and efficiency improvements in conventional agriculture are beginning to fall short of demand for food. Future farming methods are looking toward algal production as they were with Chlorella in the impending food shortage of the 1950's. Race track phototropic growth on CO2 of Spirulina depends on use of cheap waste gas. Algae from heterotropic growth on corn based carbohydrates too, like Spirulina. is far too costly for fish or live stock feeds.

There is an acute need for a municipal waste process that replaces the hundred year old activated sludge process that converts carbon and nitrogen in human waste to useful fertilizer, methane and fish/livestock feed and processes the water to a purity that allows recycling it as drinking water to mitigate further ground water depletion. These things are all accomplished in this invention.

### BACKGROUND AND PRIOR ART

The prevailing municipal waste water treatment process used around the world today is known as the Activated Sludge process. It was invented in the UK in 1914 and uses a mixed culture of microorganisms to aerobically grow in a batch open reactor thereby consuming organic matter in the sewage as well as nitrogenous and phosphoric compounds that, if allowed to return to rivers and oceans, would cause eutrophication and injury to flora and fauna. There is no closure on reactors and the “brown floc” that typically results from contact with open air is made up of many organisms. Among them, but not limited to this group, are saphotrophic bacteria; protozoa, including amoeba, spirotrichs, peritrichs and other filter feeders; rotifers and at times undesirable sphaerotilus natus bacteria. Pathogens can grow in these flocs at will if nutrients needed are there and they happen to blow in with the unfiltered aeration from a blower. Plants that employ an anaerobic denitrification step prior to aerobic oxidation create an environment selective for cultures of genus pseudomonas a facultative bacteria that flows through the following aerobic stage and if poorly clarified and improperly disinfected the effluent will dump high levels of these known live pathogens in waterways. The denitrification step also produces a greenhouse gas 300 times as potent as carbon dioxide, nitrous oxide. Synthetic fertilizer production processes are also a major emitter of this and an organic waste treatment plant by product alternative fertilizer from municipalities should cut these emissions.

The simplest flow of the process is two stages wherein in the first stage raw sewage flows into a tank reactor of the aerated organisms and liquor that consume nutrients and is then pumped to a settling tank where the clarified supernatant is released to a river organics, nitrogenous and phosphoric compounds having been removed, while the settled solids on the bottom are removed further settled and dewatered mechanically. Next they are disposed of by incineration or landfill. A primary clarification stage may precede the bioreactor stage to remove mainly cellulosic suspended solids of feces and toilet paper. For years there were no by products from this process, only a stream of seven percent solids with negative value that required expense of further processing potentially bio-hazardous solids slurry from contact with the population, that being by further water removal and land fill. Recently the EPA has issued guidelines for use of sludge known to possess considerable pathogens to be used as fertilizer called “bio solids” if treated by heating at required temperature/time profiles to reduce but not eliminate pathogen levels to acceptable risks levels all of them being short of pasteurization criteria.

### SUMMARY OF THE PRESENT INVENTION

The present invention is a radical departure from the activated sludge process as it uses modern biochemical engineering processing that employs a selected pure culture microbial continuous processes along with modern processing equipment as shown in the flow chart of FIG. 1 to remove potential pollutants from municipal waste water while producing three valuable by products: methane, high nitrogen safe organic fertilizer and a high protein animal/fish feed. The use selected cultures of yeasts or other combinations of selected microbes to remove organics, nitrogenous and phosphoric compounds in a continuous fermentation is novel as is combining it in an overall design with an anaerobic digester to provide fuel to heat the hot water to control temperature of the fermenter in winter as well as steam for fermenter sterilization that is an integration of which there is no prior art.

In the primary settling tank influent suspended solids of feces and toilet paper at about 200 ppm containing about 40-50 percent of total influent BOD settle to the bottom as sludge and is removed to the anaerobic digester by a rake to the center sump pump which in a closed round tank sludge containing methane producing species of the archaea family derived from the human colon and in combination with other bacteria digests cellulosic compounds and other substrates through methanogenesis to continuously produce biogas and cell growth that once a shift the biomass is removed and dewatered mechanically, as overhead a tank pressure activated gas pump charges a Molecular Gate unit to purge carbon dioxide and nitrogen for high pressure storage of purified methane by simple on off pressure control at set point for the digester pressure.

The over flow from the clarifier containing 20 ppm suspended solids is fed to a splitting bowl centrifuge to remove 95% of all solids and reduce the load of fouling agents on a a tangential micro-filter (Graver Technologies Scepter Crossflow with a one micron absolute pore size with radial outward flow through a rugged ceramic membrane cast over sintered stainless steel). The flow scheme incorporates a recycle pump line through the filter to rapidly recirculate and clean the surface from blinding with solids from the overflow stream not removed from influent by sedimentation and centrifugation upstream steps. Centrifuged solids are transferred to the digester.

The resulting bacteria free filtrate without previous uneconomical heat sterilization from the tangential filter is then fed to an air agitated steam sterilized fermenter with steam from a methane fired boiler. The fermenter has a H/D ratio of 4 and airflow at one volume of sterile air (Pall 0.45 micron absolute fllters)per volume of the fermenter per minute with an overhead pressure of 5 psig at the air discharge in the dome of the closed vessel. It has an OTC (oxygen transfer capacity) of about 40 mmoles/liter/hour.

This fermentation incorporates a pure culture of a strain of Candida lipolytica yeast alone or in combination with other selected microbes from ATCC that consume the organic substrates, nitrogenous and phosphorous nutrients in filtrate that compose the other soluble half of influent BOD at about 100 to 150 ppm representing mainly rendered fats mad miscible by salts of fatty acids such as sodium stearate that are in such a low concentration that a side arm recirculating loop filter of the same Scepter Crossflow design as has to be employed to retain the yeast growth as the bioreactor volume turns over frequently and the concentration builds to a density allowing a periodic purge from a continuous centrifuge to feed a conventional drum dryer or spray dryer for a dried high protein yeast animal/fish feed. Also, another feature of this continuous fermentation is that it is automatically temperature controlled by PI control of cooling or heating water flow rate delivered by variable speed pumps to internal coils or jackets of the fermenter. Ph control by addition of sodium hydroxide or ammonia gas by an on-off controller with an add/mix time cycle is also employed. Neither of these critical environmental requirements can be achieved in the poorly mixed open pits of activated sludge systems.

The filtrate from the Graver Technologies Scepter Crossflow 3.0 micron side arm tangential filter is devoid of all particles larger than that pore size as it retains all 5 micron or larger yeast It is further pumped through a spiral wound ultrafiltration (reverse osmosis) unit with a 25 MW cut off to purify the filtrate and returned to the municipal water supply storage tanks eliminating the need for intake of ground or surface water and processing it to drinking water purity except for make up quantities in the municipal supply.

1. A novel municipal waste treatment plant design that produces methane and a bacterial biomass of dead obligate anaerobes suitable as a high nitrogen organic fertilizer by employing a continuous anaerobic digestion of cellulosic solids fed from a primary clarifier.

2. A process using a splitting bowl centrifuge and ceramic membrane coated sintered stainless steel tangential filter with reflux to remove bacteria from primary clarifier overflow and prevents potential bacteria in the nutrient influent feed from contaminating the pure culture continuous yeast fermentation.

3. A plant design incorporating a continuous pure Candid lipolytica yeast fermentation with a side arm loop incorporating a sintered stainless steel tangential filter and centrifuge for concentrating broth with periodic withdrawal once a shift and purging a sterile filtrate the the bioreactor volume turns over frequently.

4. The said fermenter having air agitation and OTE of 5 KG oxygen/kwh and OTC of 40 mmoles/liter hour and Height to Diameter (H/D) ratio of 4 unless less than 50,000 gallon in which H/D of 2.5 is employed and mechanical agitation with 15 HP/1000 gallons is also employed.

5. A final ultrafiltration (reverse osmosis) of filtrate from the Graver tangential filter on the fermenter loop in a hollow fiber or spiral wound membrane unit with a molecular cut off point similar to desalination installations of molecular weight 25 is achieved by pumping pure water through the membrane against the osmotic pressure of filtrate.
